(19) United States
(12) Patent Application Publication
(10) Pub. No.: US 2009/0253901 A1
(43) Pub. Date: Oct. 8, 2009

(54) Nanoencapsulation and Release of Nucleic Acids

(76) Inventor: Alexander Grinberg, San Francisco, CA (US)

Correspondence Address: Alexander Grinberg, 600 15th Avenue, San Francisco, CA, 94118, US

(21) Appl. No.: 12/418,590

(22) Filed: Apr. 4, 2009

Related U.S. Application Data
(60) Provisional application No. 61/042,729, filed on Apr. 5, 2008.

(51) Int. Cl. C07H 21/04

(52) U.S. Cl. 536/23.1

(57) ABSTRACT

A method for Encapsulation of nucleic acids such as DNA, RNA any other types of nucleotides via entrapping or co-precipitation in CaCO3 porous microparticles followed by polymeric shell deposition or polymer nanoparticles composite shell deposition. This method can be used for controlled delivery and release of nucleic acids.

### DETAILED DESCRIPTION OF THE INVENTION

The method of encapsulation of nucleic acids such as DNA, RNA or any types of nucleotides and its mixtures, as well as their mixture with other bioactive compounds in polyelectrolyte microcapsules utilizes porous calcium carbonate microparticles which can be fabricated on the scale of 1-10 microns with fine size distribution as microscopic supports for layer-by-layer (LbL) polyelectrolyte (PE) assembling via charge interaction of alternating positive and negative charged PEs. These PE multilayers (thickness, composition) determine shell of capsules and could tuned in permeability, functionality (optically and magnet addressing), stability and degradation. Range of used PEs involved synthetic and natural charged polymers (including polysaccharides and polypeptides).

Two different methods can be used to prepare nucleic acid-loaded CaCO3 microparticles:
(i) physical adsorption—adsorption of nucleic acids from the solutions onto preformed porous CaCO3 microparticles, and
(ii) co-precipitation—nucleic acids are captured by CaCO3 microparticles in the process of growth from the mixture of aqueous solutions of CaCl2 and Na2CO3. Amount of encapsulated materials could reach 100 μg per 1 mg of CaCO3 and encapsulation efficiency close to 100%.

The procedure of encapsulation is very mild and involves no chemical treatment, but only physical capturing. CaCO3 particles could be dissolved at very mild condition leaving nucleic acids inside capsules. No change of any secondary structure of DNA or RNA or their conformation.

The advantage of the suggested approach is the possibility to control easily the concentration of amount of nucleic acids inside the microcapsules and to tune release (action) time.

Cost of technology is rather low and includes mainly costs of degradable polymers, compounds to be encapsulated, and involved man-power. This process is easily done in lab scale up-to a volume in liters, but can be scaled-up to a larger amount.

What is claimed is:

1. A method of incorporating nucleic acids such as DNA, RNA or other nucleotides into CaCO3 microparticles

2. The method of claim 1 where said nucleic acid penetrates into pores through physical adsorption on pre-formed porous CaCO3 microparticles

3. The method of claim 1 where said nucleic acid co-precipitates into CaCO3 microparticles by means of mixing said nucleic acid, NaCO3 and CaCl2 to form particles of CaCO3 that contain nucleic acids in amount up to 20 w. %

4. The method of claim 3 where particle size of formed CaCO3 particles with nucleic acids can be controlled by stirring speed, shape of vessel and/or volume added while mixing nucleic acids, NaCO3 and CaCl2.

5. The method of claim 1, claim 2, and/or claim 3 where said nucleic acid are combined with other nucleic acids and/or other additives such as proteins, promoters of DNA expression, and co-factors for multiple capsule composition.

6. The method of claim 1 and/or claim 5 where a polymer shell with defined properties such as thickness, compatibility, degradation and other tailored functionality—such as magnetic or fluorescent activation—is assembled over said CaCO3 particles containing nucleic acids or a combination of nucleic acids with other bioactive components by means of layer-by-layer assembly of polyelectrolytes, interfacial adsorption, interfacial complexation, surface induced polymer synthesis, surface induced polymer precipitation or a combined approach of thereof.

7. The method of claim 6 where extraction of CaCO3 via Ca-chelating agents or lowing pH leads to the formation of purely polymeric capsules containing encapsulated nucleic acids or its combination with other components in defined amounts, thus leading to w. % of nucleic acids up to 80%

8. The method of claim 6 where said polymer shell controlling nucleic acid release is engineered in a way that allows portion-like release of its contents such as nucleic acids or other materials so that different sorts of capsules can release said nucleic acids or other materials at different times.

9. The method of claim 6 where said polymer shell is engineered in a way to response to the presence of particular compounds including pH value and ions or be digestive to certain enzymes thus inducing the release of said nucleic acids and other contents.

10. The combination of claims 7, 8, and/or 9 in any permutation

11. The method of claim 5 and/or claim 10 where said capsules can be induced via spraying/inhalation to patient.

12. The method of claim 5 and/or claim 10 where said capsules can be induced via subcutaneous injection.
